(19) United States
(12) Patent Application Publication
(10) Pub. No.: US 2025/0064423 A1
(43) Pub. Date: Feb. 27, 2025

(54) DEVICES, SYSTEMS AND METHODS FOR DETECTING AND ANALYZING SOUNDS FROM A SUBJECT'S BODY

(71) Applicant: CARDIOKOL LTD, Airport City, Lod (IL)

(72) Inventors: Alon David GOREN, Airport City (IL); Amir BEKER, Airport City (IL); Yirmi HAUPTMAN, Rishon LeZion (IL); Eli ATTAR, Airport City (IL); David ATTAR, Airport City (IL)

(73) Assignee: CARDIOKOL LTD, Airport City, Lod (IL)

(21) Appl. No.: 18/726,449

(22) PCT Filed: Jan. 2, 2023
(86) PCT No.: PCT/IL2023/050006

§ 371 Date: Jul. 3, 2024

Related U.S. Application Data
(60) Provisional application No. 63/295,976, filed on Jan. 3, 2022.

(51) Int. Cl. A61B 7/04; A61B 7/00; G10L 25/66

(52) U.S. Cl. CPC A61B 7/04; A61B 7/003; G10L 25/66

(57) ABSTRACT

Devices, systems and methods for detecting and analyzing sounds from the subject's body are disclosed.

### FIELD OF THE INVENTION

The present invention relates to the field of devices for detecting sounds from a subject's body, and more particularly, to wearable devices thereof.

### BACKGROUND OF THE INVENTION

Continuous, long-term detection and analysis of sounds from within a subject's body may provide information concerning biomarkers indicative of a health/physical/fitness-related/wellness-related condition of the subject. Moreover, simultaneous, continuous and long-term detection and analysis of sounds generated by different organs of the subject's body may provide new information concerning correlation between functions of these organs to further enhance the information concerning the subject's health/physical/fitness-related/wellness-related condition.

### SUMMARY OF THE INVENTION

Some embodiments of the present invention may provide a device for recording and detecting sounds from a subject's body, the device may include: a support configured to be removably attached to a subject's body or a subject's clothing; an acoustic sensor connected to the support and configured to detect sounds from within the subject's body and generate an output signal; an acoustic waveguide connected to the support and configured to guide the sounds from within the subject's body to the acoustic sensor; a digital storage unit connected to the support; and a processor connected to the support and configured to: receive the output signal, and at least one of: save at least a portion of the output signal in the digital storage unit, preprocess the output signal by detecting one or more subsets of data values in the output signal indicative of one or more abnormal/pathological sound patterns and save only the detected one or more subsets of data value in the digital storage unit, and analyze the output signal to detect one or more abnormal/pathological biomarkers indicative of a health/physical/fitness-related/wellness-related condition of the subject and save information related to the detected abnormal/pathological biomarkers in the digital storage unit.

In some embodiments, the acoustic sensor is configured to detect sounds of a patterns type defined in frequency bands and time-domain characteristics so as to detect sounds generated by different organs or processes of the subject's body.

In some embodiments, the acoustic sensor is configured to detect sounds of a patterns type defined in frequency bands and time-domain characteristics so as to detect sounds generated by a specific organ or a specific subgroup of organs of the subject's body.

In some embodiments, the acoustic sensor is configured to detect sounds associated with subject's speech or sounds being byproducts of subject's speech.

In some embodiments, the acoustic sensor is configured to detect sounds of a predefined narrow frequency range so as to detect sounds generated by a specific organ or a specific subgroup of organs of the subject's body.

In some embodiments, wherein the acoustic sensor is configured to detect subject's speech.

In some embodiments, the device includes two or more acoustic sensors and two or more acoustic waveguides, each of the two or more acoustic waveguides for one of the two or more acoustic sensors.

In some embodiments, the two or more acoustic sensors are configured to detect sounds of the same frequency range.

In some embodiments, each of the two or more acoustic sensors is configured to detect sounds of a different frequency range as compared to other acoustic sensors of the two or more acoustic sensors.

In some embodiments, wavelength ranges of the two or more acoustic sensors partly overlap with each other.

In some embodiments, the two or more acoustic sensors are configured to detect sounds arriving from the same direction/location from within the subject's body.

In some embodiments, each of two or more acoustic sensors is configured to detect sounds arriving from a different direction/location from within the subject's body as compared to other acoustic sensors of the two or more acoustic sensors.

In some embodiments, the processor is configured to detect at least one of the one or more abnormal/pathological sound patterns and the one or more abnormal/pathological biomarkers based on normal sound patterns and normal biomarkers, respectively.

In some embodiments, the normal sound patterns and the normal biomarkers are subject-specific and are predefined based on accumulated sound data collected from the subject.

In some embodiments, the normal sound patterns and the normal biomarkers are specific to a population or subpopulation to which the subject being monitored belongs and are predefined based on accumulated sound data collected from a plurality of individuals belonging to the population or subpopulation.

In some embodiments, the processor is configured to detect the one or more abnormal/pathological biomarkers indicative of the health condition of the subject using one or more pre-trained machine learning models.

In some embodiments, the acoustic sensor is configured to continuously detect sounds from within the subject's body.

In some embodiments, the processor is configured to control the acoustic sensor to detect sounds from within the subject's body during predetermined time intervals according to a predetermined time schedule.

In some embodiments, the processor is configured to update the time schedule based on at least one of occurrence and duration of at least one of the one or more abnormal/pathological sound patterns and the one or more abnormal/pathological biomarkers in the output signal.

In some embodiments, the device includes a communication unit connected to the support and configured to transmit data from the digital storage unit to a remote storage device or a remote computing device or remote alarming device.

In some embodiments, the communication unit is configured to transmit the data on demand.

In some embodiments, the communication unit is configured to transmit to a remote computing device a notification indicative of the detection of at least one of the one or more abnormal/pathological sound patterns and the one or more abnormal/pathological biomarkers that require immediate attention.

In some embodiments, the device includes a notification unit connected to the support and configured to generate one or more notifications indicative the detection of at least one of the one or more abnormal/pathological sound patterns and the one or more abnormal/pathological biomarkers that require immediate attention.

In some embodiments, the notification unit is configured to generate at least one of one or more visual notifications, one or more sound notifications and one or more mechanical notifications.

In some embodiments, the processor is configured to perform a sound detection test upon attachment of the device to the subject's body or the subject's clothing and initiation thereof, the sound detection test includes: analyzing the output signal from the acoustic sensor, and determining whether or not the sounds from within the subject's body are being properly detected by the acoustic sensor.

In some embodiments, upon determination of improper detection of the sounds, the communication unit is configured to transmit a respective notification to a remote computing device, wherein the respective notification includes instructions describing how to change a location of the device on the subject's body so as to cause the device to properly detect the sounds from within the subject's body.

In some embodiments, upon determination of improper detection of the sounds, the notification unit is configured to generate respective at least one of one or more visual notifications, one or more sound notifications and one or more mechanical notifications.

In some embodiments, the device includes one or more additional sensors connected to the support and configured to generate one or more additional sensor output signals.

In some embodiments, the device includes a power source connected to the device and configured to supply power to electronic components of the device.

In some embodiments, the device includes a frame connected to electronic components of the device and configured to removably connect the electronic components of the device to the support.

In some embodiments, the device includes a covering configured to be removably connected to the support and cover components of the device and accommodate the components between the support and the covering.

In some embodiments, the device includes a clip connected to the support and configured, when actuated, to push the acoustic sensor and the acoustic waveguide towards the support.

In some embodiments, the acoustic sensor includes a piezo electric element within a housing having the waveguide as one of the surfaces of the housing.

In some embodiments, the device includes at least one gel pad acoustically coupling the piezo electric element and the waveguide.

In some embodiments, the acoustic sensor includes a microphone or within a housing having the waveguide as one of the surfaces of the housing.

Some embodiments of the present invention may provide a system for detecting sounds from a subject's body, the system includes: a swallowable capsule including an acoustic transducer configured to generate a sound signal after the swallowable capsule has been swallowed by the subject; and the device according to any one of claims 1-30, wherein the acoustic sensor of the device is configured to detect the sound signal from within the subject's body and generate the output signal further based on the detected sound signal.

In some embodiments, the swallowable capsule further includes a capsule acoustic sensor configured to detect sounds from within the subject's body and generate a capsule output signal.

In some embodiments, the swallowable capsule further includes a transmitter to transmit the capsule output signal, and wherein the communication unit of the device is configured to receive the capsule output signal.

Some embodiments of the present invention may provide a kit including two or more devices as described hereinabove.

Some embodiments of the present invention may provide a method of averaging a signal, the method may include: receiving, by a computing device, an output signal being generated by a sensor; detecting repetitive portions in the output signal; applying one or more iterations of an average function on the repetitive portions to provide averaged repetitive portions; determining, for each of the one or more iterations, based on the averaged repetitive portions, whether or not the averaged repetitive portions meet a condition; and terminating the respective interaction upon the determination that the averaged repetitive portions meet the predefined condition.

Some embodiments may include determining, for each of the one or more iterations, a signal to noise ratio (SNR) value in the averaged repetitive portions of the output signal and terminating the respective iteration if the SNR value has reached a specified SNR value.

Some embodiments may include: determining, for each of the one or more iterations, a number of the repetitive portions or the averaged repetitive portions in the output signal, and terminating the respective iteration if the number of the repetitive portions or the averaged repetitive portions has reached a specified number of repetitive portions.

In some embodiments, the specified number of repetitive portions is preset or determined based on an average number of repetitive portions in the output signal over a specified time interval.

Some embodiments may include: determining, for each of the one or more iterations, a cross-correlation value between the averaged repetitive portions and a reference signal, and terminating the respective iteration if the cross-correlation value has reached a specified cross-correlation value.

In some embodiments, the specified cross-correlation value is preset or determined based on an average cross-correlation value in the output signal over a specified time interval.

In some embodiments, each of the averaged repetitive portions includes a first section having data values that are above a preset value and a second section having data values that are below the present value, and wherein the method may include: determining, for each of the one or more iterations, a SNR value of the second sections of the averaged repetitive portions, and terminating the respective iteration if the SNR value of the second sections of the averaged repetitive portions has reached a specified SNR value.

In some embodiments, the specified SNR value is preset or determined based on an average SNR value in the output signal over a specified time interval.

Some embodiments may include applying the average function on a specified number of the repetitive portions of the output signal.

In some embodiments, the specified number of the repetitive portions is preset or determined based on a preset SNR value.

Some embodiments may include determining, based on the averaged repetitive portions, one or more subsets of data values indicative of one or more sound patterns being detected from within the subject's body.

Some embodiments may include analyzing at least one of the averaged repetitive portions or the one or more sound patterns to detect one or more abnormal/pathological biomarkers indicative of a health/physical/fitness-related/wellness-related condition of the subject.

Some embodiments of the present invention may include a method of analyzing a signal, the method may include: detecting repetitive portions in the output signal; detecting repetitive portions in the output signal; subtracting the repetitive portions from the output signal to provide non-repetitive portions; and at least one of: determining, based on the non-repetitive portions, one or more subsets of data values indicative of one or more abnormal/pathological sound patterns being detected from within the subject's body; or analyzing the non-repetitive portions and/or the one or more sound patterns to detect one or more biomarkers indicative of a health/physical/fitness-related/wellness-related condition of the subject.

Some embodiments may include: applying one or more iterations of an average function on the non-repetitive portions to provide averaged non-repetitive portions; determining, for each of the one or more iterations, based on the averaged non-repetitive portions, whether or not the averaged non-repetitive portions meet a predefined condition; and terminating the respective interaction upon the determination that the averaged non-repetitive portions meet the predefined condition.

Some embodiments may include at least one of: determining, based on the averaged non-repetitive portions, the one or more subsets of data values indicative of one or more sound patterns being detected from within the subject's body; or analyzing the averaged non-repetitive portions and/or the one or more sound patterns to detect the one or more biomarkers indicative of a health/physical/fitness-related/wellness-related condition of the subject.

Some embodiments of the present invention may provide a method of detecting and analyzing sounds from within two or more locations within a subject's body, the method may include: detecting, by a first acoustic sensor, sounds from a first location within the subject's body and generating a first output signal related thereto; detecting, by a second acoustic sensor, sounds from a second location within the subject's body and generating a second output signal related thereto; determining, by a computing device, based on the first output signal, a subset of data values indicative of a series of cues or patterns of sounds being detected by the first acoustic sensor; synchronizing the second output signal with the first output signal based on the subset of data values indicative of the series of sound cues or sounds patterns being detected from the first location within the subject's body; determining, based on the synchronized second output signal, one or more subsets of data values indicative of one or more patterns of sound being detected by the second acoustic sensor.

Some embodiments may include determining, based on the first output signal, one or more subsets of data values indicative of one or more patterns of sounds being detected by the first acoustic sensor.

Some embodiments may include analyzing at least one of the first output signal or the one or more patterns of sounds being detected by the first acoustic sensor to determine one or more biomarkers indicative of a health/physical/fitness-related/wellness-related condition of the subject.

Some embodiments may include analyzing at least one of the second output signal or the one or more patterns of sounds being detected by the second acoustic sensor to determine one or more biomarkers indicative of a health/physical/fitness-related/wellness-related condition of the subject.

Some embodiments may include: determining a correlation between (i) the one or more patterns of sounds being detected by the first acoustic sensor and (ii) the one or more patterns of sound being detected by the second acoustic sensor; and determining, based on the correlation, one or more biomarkers indicative of a health/physical/fitness-related/wellness-related condition of the subject.

Some embodiments may include: measuring, by a third non-acoustic sensor, a parameter of the subject's body and generating a third output signal related thereto; determining a correlation between at least one of (i) the one or more patterns of sounds being detected by the first acoustic sensor and (ii) the one or more patterns of sound being detected by the second acoustic sensor, (iii) the one or more parameter patterns being measured by the third non-acoustic sensor, (iv) or any combination thereof; determining, based on the correlation, one or more biomarkers indicative of a health/physical/fitness-related/wellness-related condition of the subject.

Some embodiments of the present invention may provide a method of determining one or more biomarkers indicative of a health condition of a subject based on an acoustic sensor and a non-acoustic sensor, the method may include: detecting, by a first acoustic sensor, sounds from a predetermined location within the subject's body and generating a first output signal related thereto; determining, by a computing device, based on the first output signal, one or more incident events associated with a health condition of a subject; measuring, by a second non-acoustic sensor, one or more parameters associated with the health condition of the subject; and determining, based on the one or more determined incident events and the one or more measured parameters, one or more biomarkers indicative of the health condition of the subject.

Some embodiments may include determining, based on the one or more incident events and the one or more measured parameters, a cumulative load of the incident events.

In some embodiments, the first acoustic sensor detecting sounds from at least a portion of the subject's heart and the second non-acoustic sensor measuring a concentration of plasma lactate of the subject.

In some embodiments, the one or more incident events includes atrial fibrillation (AF) events and the health condition of the subject includes a cumulative load of the AF events.

Some embodiments of the present invention may include a method of detecting and analyzing sounds from a subject's joint, the method may include: detecting, by an accelerometer sensor, an acceleration of a subject's joint and generating a first output signal related thereto; detecting, by an acoustic sensor, sounds from the subject's joint and generating a second output signal related thereto; determining, by a computing device, based on the first output signal, a subset of data values indicative of a series of cues or patterns of sounds being detected by the accelerometer sensor; synchronizing the second output signal with the first output signal based on subset of data values indicative of a series of cues or patterns of sounds being detected by the accelerometer sensor; determining, based on the synchronized second output signal, one or more patterns of the sounds being detected from the subject's joint.

Some embodiments may include determining, based on at least one of the first output signal, the synchronized second output signal, one or more determined patterns of the sounds or any combination thereof, one or more biomarkers indicative of the health condition of the subject.

Some embodiments of the present invention may include a method of detecting sounds from a subject's body based on an external acoustic sensor and a swallowable capsule, the method may include: generating a sound signal by an acoustic transducer of a swallowable capsule after the swallowable capsule has been swallowed by the subject; detecting, by one or more acoustic sensors being placed on or a vicinity to a subject's body, the sound signal generated by the acoustic transducer of the swallowable capsule from within the subject's body and generating one or more output signals related thereto; determining, based on the one or more output signals, information concerning tissues through which the sound signal has passed.

Some embodiments of the present invention may provide a method of analyzing a signal indicative of sounds detected from within a subject's body, the method may include, using a computing device operating a processor: receiving an output signal generated by a sensor, the output signal being indicative of sounds detected from within a subject's body; detecting repetitive portions in the output signal; subtracting the repetitive portions from the output signal to provide non-repetitive portions; and determining, based on the non-repetitive portions, one or more subsets of data values indicative of one or more abnormal/pathological sound patterns detected from within the subject's body.

Some embodiments may include, based on at least one of the non-repetitive portions and the one or more abnormal/pathological sound patterns, detecting one or more biomarkers indicative of a health/physical/fitness-related/wellness-related condition of the subject.

Some embodiments may include: applying one or more iterations of an average function on the non-repetitive portions to provide averaged non-repetitive portions; determining, for each of the one or more iterations, based on the averaged non-repetitive portions, whether or not the averaged non-repetitive portions meet a predefined condition; and terminating the respective interaction upon the determination that the averaged non-repetitive portions meet the predefined condition.

Some embodiments may include determining the one or more subsets of data values indicative of the one or more abnormal/pathological sound patterns based on the averaged non-repetitive portions.

Some embodiments may include detecting the one or more biomarkers indicative of the health/physical/fitness-related/wellness-related condition of the subject based on at least one of the averaged non-repetitive portions and the one or more abnormal/pathological sound patterns.

Some embodiments may include applying one or more iterations of an average function on the repetitive portions to provide averaged repetitive portions; determining, for each of the one or more iterations, based on the averaged repetitive portions, whether or not the averaged repetitive portions meet a condition; and terminating the respective interaction upon the determination that the averaged repetitive portions meet the predefined condition.

Some embodiments may include determining, for each of the one or more iterations, a signal to noise ratio (SNR) value in the averaged repetitive portions of the output signal and terminating the respective iteration if the SNR value has reached a specified SNR value.

Some embodiments may include: determining, for each of the one or more iterations, a number of the repetitive portions or the averaged repetitive portions in the output signal, and terminating the respective iteration if the number of the repetitive portions or the averaged repetitive portions has reached a specified number of repetitive portions. The specified number of repetitive portions may be preset or determined based on an average number of repetitive portions in the output signal over a specified time interval.

Some embodiments may include: determining, for each of the one or more iterations, a cross-correlation value between the averaged repetitive portions and a reference signal, and terminating the respective iteration if the cross-correlation value has reached a specified cross-correlation value. The specified cross-correlation value may be preset or determined based on an average cross-correlation value in the output signal over a specified time interval.

Each of the averaged repetitive portions includes a first section having data values that are above a preset value and a second section having data values that are below the present value, and some embodiments may include: determining, for each of the one or more iterations, a SNR value of the second sections of the averaged repetitive portions, and terminating the respective iteration if the SNR value of the second sections of the averaged repetitive portions has reached a specified SNR value. The specified SNR value may be preset or determined based on an average SNR value in the output signal over a specified time interval.

Some embodiments may include applying the average function on a specified number of the repetitive portions of the output signal. The specified number of the repetitive portions may be preset or determined based on a preset SNR value.

Some embodiments may include determining the one or more subsets of data values indicative of the one or more abnormal/pathological sound patterns based on the averaged repetitive portions.

Some embodiments may include detecting one or more abnormal/pathological biomarkers indicative of a health/physical/fitness-related/wellness-related condition of the subject based on at least one of the averaged repetitive portions and the one or more abnormal/pathological sound patterns.

Some embodiments may include generating a notification indicative of the one or more sound patterns detected from within the subject's body.

Some embodiments may include transmitting a notification indicative of the one or more sound patterns detected from within the subject's body to a remote device.

Some embodiments may include generating a notification indicative of the one or more biomarkers indicative of the health/physical/fitness-related/wellness-related condition of the subject.

Some embodiments may include transmitting a notification indicative of the one or more biomarkers indicative of the health/physical/fitness-related/wellness-related condition of the subject to a remote device.

Some embodiments of the present invention may provide a computing device which may include a memory and a processor configured to perform operations described hereinabove.

### BRIEF DESCRIPTION OF THE DRAWINGS

For a better understanding of embodiments of the invention and to show how the same can be carried into effect, reference will now be made, purely by way of example, to the accompanying drawings in which like numerals designate corresponding elements or sections throughout.

In the accompanying drawings:

FIGS. 1A, 1B, 1C and 1D are schematic illustrations of a device for detecting sounds from a subject's body, according to some embodiments of the invention;

FIG. 2 is a schematic illustration of a system for detecting sounds from a subject's body, according to some embodiments of the invention;

FIG. 3 is a schematic illustration of a device for detecting sounds from a subject's body and an array of acoustic sensors connectable to the device, according to some embodiments of the invention;

FIGS. 4A-4C are schematic illustrations of a piezoelectric element within a housing serving as the acoustic sensor according to some embodiments of the invention;

FIG. 4D shows a non-limiting example of the piezoelectric element whose corners rest on supporting sections of the housing, according to some embodiments of the invention;

FIG. 5 is a flowchart of a method of averaging a signal, according to some embodiments of the invention;

FIG. 6 is a flowchart of a method of analyzing a periodic or quasiperiodic signal, according to some embodiments of the invention;

FIG. 7 is a flowchart of a method of detecting and analyzing sounds from within two or more locations within a subject's body, according to some embodiments of the invention;

FIG. 8 is a flowchart of a method of determining one or more biomarkers indicative of a health condition of a subject based on an acoustic sensor and a non-acoustic sensor, according to some embodiments of the invention;

FIG. 9 is a flowchart of a method of detecting and analyzing sounds from a subject's joint, according to some embodiments of the invention;

FIG. 10 is a flowchart of a method of detecting sounds from a subject's body based on an external acoustic sensor and a swallowable capsule, according to some embodiments of the present invention, according to some embodiments of the invention; and

FIG. 11 is a block diagram of an exemplary computing device which may be used with embodiments of the present invention.

It will be appreciated that, for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following description, various aspects of the present invention are described. For purposes of explanation, specific configurations and details are set forth in order to provide a thorough understanding of the present invention. However, it will also be apparent to one skilled in the art that the present invention can be practiced without the specific details presented herein. Furthermore, well known features can have been omitted or simplified in order not to obscure the present invention. With specific reference to the drawings, it is stressed that the particulars shown are by way of example and for purposes of illustrative discussion of the present invention only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention can be embodied in practice.

Before at least one embodiment of the invention is explained in detail, it is to be understood that the invention is not limited in its application to the details of construction and the arrangement of the components set forth in the following description or illustrated in the drawings. The invention is applicable to other embodiments that can be practiced or carried out in various ways as well as to combinations of the disclosed embodiments. Also, it is to be understood that the phraseology and terminology employed herein is for the purpose of description and should not be regarded as limiting.

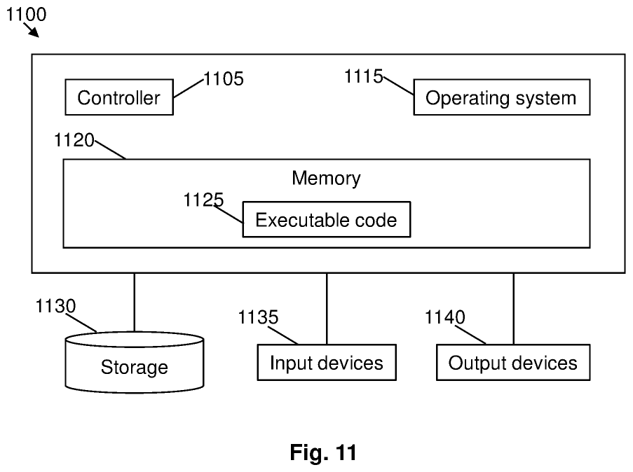

Unless specifically stated otherwise, as apparent from the following discussions, it is appreciated that throughout the specification discussions utilizing terms such as “processing”, “computing”, “calculating”, “determining”, “enhancing” or the like, refer to the action and/or processes of a computer or computing system, or similar electronic computing device (e.g., such as computing device 1100 described below with respect to FIG. 11), that manipulates and/or transforms data represented as physical, such as electronic, quantities within the computing system's registers and/or memories into other data similarly represented as physical quantities within the computing system's memories, registers or other such information storage, transmission or display devices. Any of the disclosed modules or units can be at least partially implemented by a computer processor.

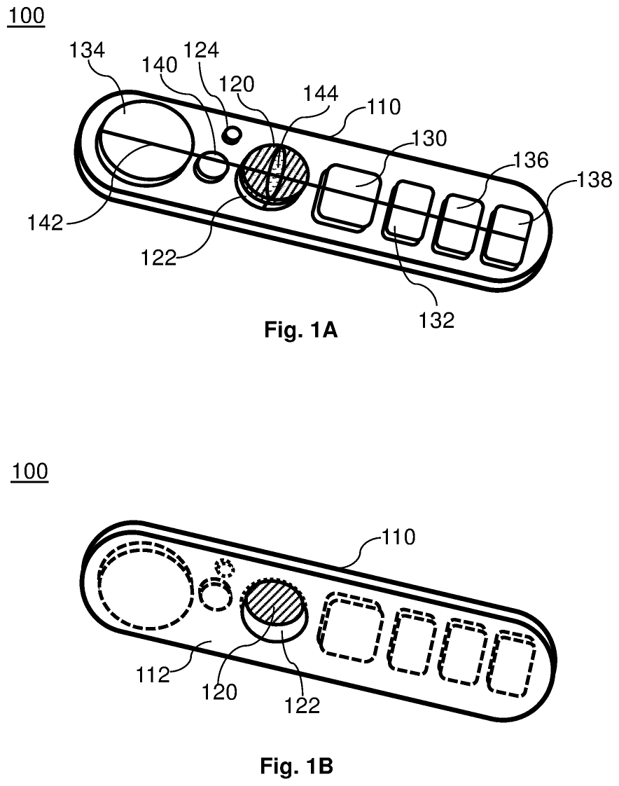

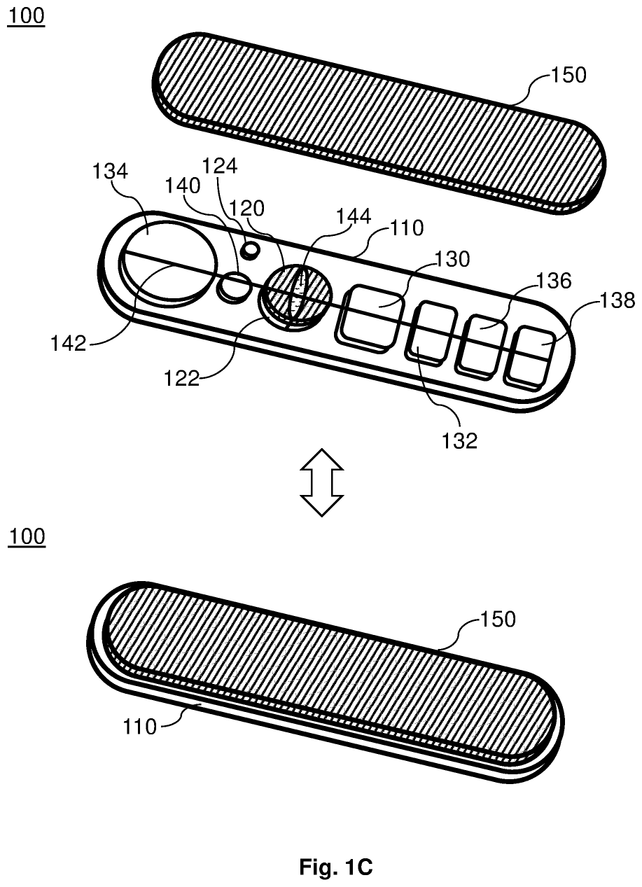

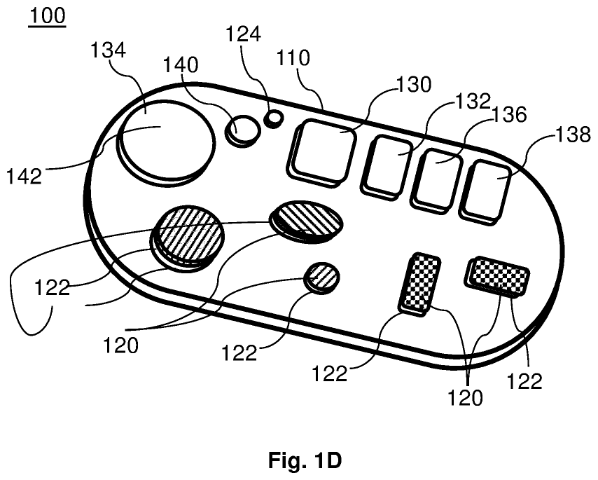

Reference is now made to FIGS. 1A, 1B, 1C and 1D, which are schematic illustrations of a device 100 for detecting sounds from a subject's body, according to some embodiments of the invention. FIGS. 1A and 1B schematically show different views of device 100.

Device 100 may include a support 110. In some embodiments, support 110 may be flat (or substantially flat) as depicted in the Figures. Flat may mean not having protrusions or recesses. In some embodiments, support 110 may be flexible and still remain flat. In some embodiments, support 110 may be removably attachable to a subject's body. For example, support 110 may include a flat sticky surface 112 to removably stick support 110 to the subject's body. Support 110 may be attached to the subject's body using components such as clip, belt, bio-compatible sticker or glue, pressure grip or any other suitable component known in the art.

In some embodiments, support 110 may be removably attachable to a subject's clothing. For example, support 110 may include one or more fasteners (e.g., such as tape, scotch tape, stitch, stitched pocket, etc.) to removably attach support 110 to subject's clothing. Support 110 may have different geometric shapes.

Device 100 may include an acoustic sensor 120. Acoustic sensor 120 may be connected to support 110. Acoustic sensor 120 may detect sounds from within the subject's body. In some embodiments, acoustic sensor 120 may detect sounds from within the subject's body in a vicinity of acoustic sensor 120. Acoustic sensor 120 may generate an output signal indicative of the detected sounds. Acoustic sensor 120 may have different geometric shapes. Acoustic sensor 120 may be of various types, such as, for example directional acoustic sensor, omnidirectional acoustic sensor, cardioid acoustic sensor, etc. In some embodiments, acoustic sensor 120 may include a microphone. In some embodiments, acoustic sensor 120 may include a hydrophone. In some embodiments, acoustic sensor 120 may include piezoelectric element. For example, the piezoelectric element may include a piezoelectric film or crystal such as polyvinylidene fluoride (PVDF). In some embodiments, acoustic sensor 120 may include a supportive case. In some embodiments, acoustic sensor 120 may be provided without a supportive case.

In some embodiments, device 100 may include an acoustic waveguide 122. Acoustic waveguide 122 may be connected to support 110, for example between support 110 and acoustic sensor 120. Acoustic waveguide 122 may guide the sounds from the subject's body to acoustic sensor 120. According to some embodiments, acoustic waveguide 122 may isolate sounds from the subject's body from ambient sounds. Acoustic waveguide 122 may achieve this isolation by restricting the transmission of energy (e.g. sounds from the subject's body) to one direction, which may reduce losses in the energy otherwise caused by interaction with ambient sources in other directions. In some embodiments, acoustic waveguide 122 may include a sleeve. The sleeve of acoustic waveguide 122 may, for example, be made from a polymer or a metal. Acoustic waveguide 122 may have different geometric shapes. For example, acoustic waveguide 122 may have circular, elliptical, rectangular or other any shape.

In some embodiments, a gel having desired acoustic properties may be used to enhance the acoustic coupling of acoustic sensor 120 and acoustic waveguide 122 to the subject's body. For example, the gel may have an acoustic impedance similar to human tissue. The gel may displace air between the subject's body and the acoustic sensor 120 and acoustic waveguide 122, thereby creating a vacuum effect to improve signal acquisition. According to some embodiments, a gel pad is included in the housing which couples a piezoelectric element to the housing.

In some embodiments, the gel pad acoustically coupling the piezo electric element and the waveguide can be made of one of: PZT film/crystal/ceramic/PVDF.

In some embodiments, device 100 may include an acoustic membrane (not shown) to couple the detected sounds from within the subject's body to acoustic sensor 120. In some embodiments, the acoustic membrane is instead of acoustic waveguide 122. In some embodiments, the acoustic membrane is in addition to acoustic waveguide 122.

In various embodiments, device 100 may include a seal and/or insulator 126 (e.g. schematically shown in FIG. 1A by dashed circle). Seal and/or insulator 126 may, for example, include sleeve, a coating layer or material or any other suitable component or device known in the art. For example, if seal and/or insulator 126 is a gel-like material, acoustic sensor 120 may be immersed in the material. Seal and/or insulator 126 may, for example, reduce noise and/or increase the signal-to-noise ratio (SNR) of signals generated by acoustic sensor 120. In some embodiments, seal and/or insulator 126 may be used instead of acoustic waveguide 122. In some embodiments, seal and/or insulator 126 may be used in addition to acoustic waveguide 122.

In some embodiments, acoustic sensor 120 may detect sounds of a predefined wide frequency range. For example, acoustic sensor 120 may be capable of sensing sounds generated by different organs of the subject's body (e.g., heart, lungs, large intestine, etc.). For example, the wide frequency range may include 0.1 Hz to 40 kH. In some embodiments, different processing type of the output signal may be required for sound frequency ranges.

In some embodiments, acoustic sensor 120 may detect sounds of a predefined narrow frequency range. For example, acoustic sensor 120 may be capable of sensing sounds generated by a specific organ or by a subgroup of organs of the subject's body. For example, the narrow range may include any sub-band of the wide frequency range of 0.1 Hz to 40 kH, for example, 0.1 Hz to 20 KHz, 10 Hz to 2000 Hz, etc.

In some embodiments, acoustic sensor 120 may detect subject's speech. In some embodiments, acoustic sensor 120 may detect sounds caused by subject's breath. In some embodiments, acoustic sensor 120 may detect sounds caused by subject's cough.

In some embodiments, device 100 may include two or more acoustic sensors 120. In some embodiments, device 100 may include two or more acoustic waveguides 122 each for one of the acoustic sensors 120. In some embodiments, two or more acoustic sensors 120 may detect sounds of the same frequency range (e.g., the same wide frequency range or the same narrow frequency range). In some embodiments, some of two or more acoustic sensors 122 may detect sounds of a different frequency range as compared to other acoustic sensors of two or more acoustic sensors 122. For example, the frequency range of each of two or more acoustic sensors 122 may be selected based on a specific organ or a subgroup of organs of the subject's body to be sensed with the respective acoustic sensor. For example, a first acoustic sensor may be capable of detecting sounds from a subject's heart and operate in a first frequency range of 20-200 Hz, a second acoustic sensor may be capable of detecting sounds from subject's lungs and operate in a second frequency range of 25-1500 Hz. In some embodiments, the wavelength ranges of two or more acoustic sensors 122 may partly overlap with each other. In some embodiments, two or more acoustic sensors 120 may be configured to detect sounds arriving from the same direction from within the subject's body. In some embodiments, some of two or more acoustic sensors 120 may be configured to detect sounds arriving from a different direction from within the subject's body as compared to other acoustic sensors of two or more acoustic sensors 120. In some embodiments, some of two or more acoustic sensors 120 may have different shape as compared to other acoustic sensors of two or more acoustic sensors 120. In some embodiments, some of two or more acoustic sensors 120 may be of a different type as compared to other acoustic sensors of two or more acoustic sensors 120. For example, FIG. 1D shows an example of device 100 having multiple acoustic sensors 120.

Having two or more acoustic sensors 120 within device 100 may have several advantages. For example, when using two or more acoustic sensors 120, two or more different organs/processes within the subject's body can be simultaneously and/or consequently monitored and correlation between these processes can be determined and new biomarkers may be created. In another example, when using two or more acoustic sensors 120, it is possible to monitor sounds generated by, e.g., a blood flow at different locations within the subject's body. In another example, when using two or more acoustic sensors 120, each of the two or more acoustic sensors 120 may be directed to a different direction as compared to other acoustic sensors. In another example, when using two or more acoustic sensors 120, the respective output signals may be used to determine and separate the sound sources within the subject's body. In another example, when using two or more acoustic sensors 120, a signal-to-noise ratio (SNR) of the output signals may be enhanced.

According to some embodiments, a frequency of the output signal may be modulated.

Device 100 may include electronic components such as amplifier(s), filter(s), analog-to-digital convert(s) and any other suitable electronic components known in the art.

Device 100 may include a processor 130. Processor 130 may be connected to support 110. Processor 130 may receive the output signal(s) from acoustic sensor(s) 120.

In some embodiments, processor 130 may save at least a portion of the output signal(s) in a digital storage unit 132. For example, processor 130 may compress the output signal(s) and save the compressed output signal(s) in digital storage unit 132.

In some embodiments, processor 130 may preprocess the output signal(s). In some embodiments, processor 130 may save only the preprocessed output signal(s) in digital storage unit 132. For example, processor 130 may detect one or more subsets of data values in the output signal(s) indicative of one or more abnormal/pathological sound patterns and save in digital storage unit 132 only the detected subset(s) of data values.

In some embodiments, processor 130 may analyze the output signal(s) to detect one or more abnormal/pathological biomarkers indicative of a health/physical/fitness-related/wellness-related condition of the subject. In some embodiments, processor 130 may save in digital storage unit 132 information related to detected abnormal/pathological biomarker(s). In some embodiments, processor 130 may detect the abnormal/pathological biomarker(s) in the output signal(s) using one or more pre-trained artificial intelligence (AI) models. In some embodiments, processor 130 may detect the abnormal/pathological biomarker(s) in the output signal(s) using one or more pre-trained artificial machine learning models.

In some embodiments, processor 130 may detect one or more abnormal/pathological biomarkers indicative of the health condition of the subject based on the detected subject's speech. For example, processor 130 may analyze the detected subject's speech using one or more AI methods and/or one or more machine learning methods.

In various embodiments, processor 130 may detect the abnormal/pathological sound pattern(s) and/or the abnormal/pathological biomarker(s) in the output signal(s) based on normal sound pattern(s) and/or normal biomarker(s), respectively. In various embodiments, the normal sound pattern(s) and/or the normal biomarker(s) may be subject specific. For example, the normal sound pattern(s) and/or the normal biomarker(s) may be defined based on accumulated sound data collected from that particular subject. In various embodiments, the normal sound pattern(s) and/or the normal biomarker(s) may be specific to a population or a subpopulation to which the subject being monitored belongs. For example, the normal sound pattern(s) and/or the normal biomarker(s) may be defined based on accumulated sound data collected from a plurality of individuals belonging to this particular population or subpopulation.

In some embodiments, acoustic sensor(s) 120 may be configured to continuously detect sounds from within the subject's body. In some embodiments, processor 130 may control acoustic sensor(s) 120 to detect sounds from within the subject's body during predetermined time intervals according to a predetermined time schedule. In some embodiments, processor 130 may update the time schedule based on the output signal(s). For example, processor 130 may update the time schedule based on the occurrence and/or duration of the abnormal/pathological sound pattern(s), the occurrence and/or duration of the abnormal/pathological biomarker(s) in the output signal(s), etc.

Device 100 may include a power source 134. Power source 134 may be connected to support 110. Power source 134 may supply power to components of device 100. In some embodiments, power source 134 may include one or more batteries.

In some embodiments, device 100 may include a communication unit 136. Communication unit 136 may be connected to support 110. In some embodiments, communication unit 136 may be a wireless communication unit. Wireless communication unit 136 may be, for example, near-field communication (NFC)-based unit, Bluetooth-based unit, radiofrequency identification (RFID)-based unit, etc.

Communication unit 136 may transmit data from digital storage unit 132 to a remote storage device or a remote computing device. In some embodiments, communication unit 136 may transmit the data on demand. For example, communication unit 136 may receive a transmission request signal and transmit the data upon receipt of the transmission request signal.

In some embodiments, the remote device may perform at least some of functions of processor 130 of device 100 as described herein.

In some embodiments, communication unit 136 may transmit to a remote computing device a notification indicative of the detection of the abnormal/pathological sound pattern(s) and/or the detection of abnormal/pathological biomarker(s) that require immediate attention. For example, the remote computing device may be a smartphone of the subject, appointed physician's smartphone, healthcare center's server, etc.

In some embodiments, communication unit 136 may be a wired communication unit. For example, communication unit 136 may be connected to a remote storage device or a remote computing device using a wire (e.g., universal serial bus (USB) cable, I2C, Rs232, Ethernet cable, etc.) to transmit the data from digital storage unit 132 to the remote storage device or the remote computing device.

In various embodiments, device 100 may include a remote storage unit or a remote computing unit to download and/or upload data from/to digital storage unit 132/processor 130 of device 100.

In some embodiments, device 100 may include a notification unit 138. Notification unit 138 may be connected to support 110. Notification unit 138 may generate one or more notifications indicative of, for example, the detection of the abnormal/pathological sound pattern(s) and/or the detection of abnormal/pathological biomarker(s) that require immediate attention. In some embodiments, notification unit 138 may generate one or more visual notifications. For example, notification unit 138 may include a light-emitting diode (LED) configured to generate, e.g., red light if immediate attention is required. In some embodiments, notification unit 138 may generate one or more audio notifications. For example, notification unit 138 may include a speaker configured to generate, e.g., a predefined sound if immediate attention is required. In some embodiments, notification unit 138 may include a vibrating member configured to generate vibrations if immediate attention is required. Other examples of notification units 138 are also possible.

In some embodiments, processor 130 may perform a sound detection test upon attachment of device 100 to the subject's body and initiation thereof. For example, upon attachment of device 100 to the subject's body and initiation thereof, processor 130 may analyze the output signal(s) from acoustic sensor(s) 120 to determine whether or not the sounds are being properly detected. In some embodiments, upon determination of improper detection of the sounds, communication unit 136 may transmit a respective notification to a remote computing device. For example, communication unit 136 may transmit such notification to a subject's smartphone. The notification may, for example, include instructions concerning of e.g., how to change a location of device 100 on the subject's body so as to cause device 100 to properly detect the sounds. In some embodiments, upon determination of improper detection of the sounds, notification unit 138 may generate respective one or more visual or sound notifications (e.g., as described hereinabove).

In some embodiments, device 100 may include a sound transmitter 124. Sound transmitter 124 may transmit sounds into the subject's body. Acoustic senor 122 (e.g., of device 100 or of any other suitable device similar to device 100 placed on the subject's body) may be synchronized to receive the sound transmitted by sound transmitter 124 and/or the reflected sound and generated the output signal related thereto. Processor 130 may analyze the output signal or cross-correlate the output signal with the sound transmitted the transmitter 124 (e.g., to identify changes in the signal's phase, power, spectral features, or any other suitable parameters) to determine the condition of, for example, a target tissue, organ or flow (e.g., blood, fluids, peristaltic). This may be done using, for example, a single device 100 or by two or more devices similar to device 100 and placed at different positions on or in a vicinity of the subject's body.

In some embodiments, device 100 may include one or more additional sensors 140. Additional sensor(s) 140 may be connected to support 110. Additional sensor(s) 140 may, for example, include an accelerometer, electrocardiography (ECG) sensor, photoplethysmogram (PPG) sensor, temperature sensor, moisture sensor, skin conductance sensor, etc. Additional sensor(s) 140 may generate additional output signal(s) that may be further analyzed to, for example, determine correlations between different indications related to the additional output signal(s).

In some embodiments, device 100 may be disposable. In some embodiments, support 110 and optionally waveguide 122 of device 100 may be disposable while at least electronic components of device 100 may be reusable. The electronic components of device 100 may, for example, include acoustic sensor 120, processor 130, digital storage unit 132, communication unit 136, notification unit 138 and power source 140. For example, device 100 may include a frame 142 connected to the electronic components of device 100 and configured to removably connect the electronic components to support 110.

In some embodiments, device 100 may include a clip 144. Clip 144 may be connected to support 110. Clip 144 may be configured, when actuated, to push acoustic sensor 120 and acoustic waveguide 122 towards support 110 to provide a desired contact pressure of between acoustic waveguide 122/acoustic sensor 120 and the subject's body.

In some embodiments, device 100 may include a covering 150 configured to be connected (or removably connected) to support 110 and cover components of device 100 to thereby accommodate the components between support 110 and the covering (e.g., as shown in FIG. 1C).

Device 100 has several advantages over typical commercial electronic stethoscope devices. Device 100 may include waveguide 122 to guide sounds detected from within the subject's body to acoustic sensor 120 in contrast to typical commercial electronic stethoscope devices that typically utilize an acoustic membrane to couple the detected sounds to the acoustic sensor. Waveguide 122 occupies significantly less space as compared to the acoustic membrane. Accordingly, device 100 may have significantly smaller dimensions and weight and/or may have more acoustic sensors 120 (or additional sensors such as accelerometers 140) connected to support 110 as compared to typical commercial electronic stethoscope devices. For example, a sub-assembly of acoustic sensor 120 and waveguide 122 of device 100 may have a diameter of 0.3-0.5 cm and a height of 0.1-0.3 cm, while typical electronic stethoscope device may have a diameter of 2-4.5 cm and height of 1-2 cm. Moreover, waveguide 122 requires significantly smaller contact pressure (or requires no contact pressure at all) to efficiently guide the sounds detected from within the subject's body to the acoustic sensor, in contrast to the acoustic membrane that requires significant contact pressure to provide sufficient coupling of the detected sounds to the acoustic sensor. Accordingly, device 100 may be removably attached to the subject's body by relatively simple means, for example, using sticky flat flexible support 110 as described hereinabove. Furthermore, device 100 may remain attached to the subject's body for long periods of times (e.g., days, weeks, etc.) without causing (or substantially without causing) inconvenience to the subject.

Device 100 may be removably attached to the subject's body at various locations. For example, device 100 may be attached to the subject's chest, back, abdomen, joints, etc. The body locations for attaching device 100 may be selected based on, for example, an organ or a subgroup of organs to be sensed with device 100.

In some embodiments, device 100 may be configured to detect sounds from different portions of a specific organ of the subject's body. For example, device 100 configured to detect sounds generated by a subject's heart may include a first acoustic sensors (e.g., like acoustic sensor 120) to detect sounds generated by one or more valves of the subject's heart and a second acoustic sensor (e.g., like acoustic sensor 120) to detect cardiac murmur.

In some embodiments, device 100 may be configured to detect sounds from a subgroup of organs of the subject's body. For example, device 100 may include a first acoustic sensor (e.g., like acoustic sensor 120) to detect sounds generated by a subject's heart and one or more second acoustic sensors (e.g., like acoustic sensor 120) to detect sounds generated by subject's lungs, optionally at different locations along the lungs.

Device 100 may have different shapes. The shape of device 100 may be, for example, predefined based on an organ or a subgroup of organs to be sensed with device 100. For example, device 100 configured to detect sounds generated by a subject's large intestine may have substantially the same shape as the large intestine and may include several acoustic sensors configured to detect sounds at different locations along the large intestine.

Device 100 may be used to monitor fetal parameters such as, e.g. fetal motion, heart beat, heart rate or any other suitable fetal parameters known in the art.

One or more devices 100 may be removably attached to the subject's body for long periods of times (e.g., days, weeks, etc.) to continuously detect sounds from within the subject's body. Continuous, long-term detection and analysis of sounds from within the subject's body may provide information concerning subject's health condition. Moreover, simultaneous, continuous and long-term detection and analysis of sounds generated by different organs of the subject's body may provide new information concerning correlation between functions of these organs to further enhance the information concerning subject's health condition.

Some embodiments of the present invention may provide a kit including two or more devices (e.g., each like device 100) for detecting sounds from within the subject's body. For example, the kit may include a first device (e.g., like device 100) configured to be attached to a subject's chest to detect sounds generated by a subject's heart, and a second device (e.g., like device 100) configured to be attached to a subject's back and to detect sounds generated by subject's lungs, optionally at different locations in the lungs.

Device-based recordings, being recorded continuously over many hours and in diversified settings, may provide a much broader basis for the analysis of the recorded signals than typical sporadic or routine spot-checks. Constant use of the device may, for example, serve an essential role in dramatically increasing the accuracy of voice-based analysis, by enabling a personalized fine tuning of the features, thresholds and overall models. A combined offering of the device (e.g., for an initial period or for short periods), and, for example, voice-based monitoring of the subject, may, for example, reach substantially better degrees of clinical accuracy and usability. Using the different sensing capabilities of the device (e.g., ECG, spoken voice, body sounds including heart sounds or any other suitable sounds), and the intrinsic simultaneity of recorded data, a database of various points in the parameter space may be registered (e.g., different heart rates, breathing conditions, arrhythmias if exist, or any other suitable parameters), together with the corresponding points and areas in the feature space, of the analyzed spoken voice. Various conditions may be correlated to their corresponding voice features, thus better defining the boundaries of “normal” and “abnormal” (e.g., AF or other pathologies) sub-spaces in the feature space of the existing model. Moreover, in some embodiments, a personalized model may be constructed for each individual subject based on this analysis and distinction. Some implementations may, for example, include (i) different heart rate conditions, (ii) different breathing rate and breathing depth conditions, (iii) sinus rhythm, AF, different arrhythmias, (iv) different motion patterns of the body, including vibrations (e.g., car ride, etc.), (v) different postures of the body. One example, may, for example, include adaptive tuning of different arrhythmia states to other parameters, e.g., heart rate. In this example, a library of different heart rate values to consequent parameter values may be created, in sinus rhythm and in Afib condition (may be extended to other arrhythmias)—at 70 bpm/100 bpm/140 bpm, different “fingerprints” of voice features correspond to normal vs. the Afib condition. In another example, a cohort-dependent characteristic voice feature “fingerprints”, to enhance clinical accuracy and resolution of detection may be created. In this example, additional parameters (other than heart rate) will be considered for categorizing sub-populations, such as age group, CHA₂DS₂-VASc Score, basic voice features and others.

According to some embodiments device 100 has the form of a silicone pad with domes, with microphones inside the domes. For example, device 100 may have the form factor of a 2×2 array of four silicone hemispheres, with a microphones located in each internal cavity of the hemispheres. In some embodiments, the domes may not be perfectly spherical but may be a polygonal approximation of a hemisphere, for example a faceted 3D shape composed of 2D polygons such as triangles, squares, pentagons, hexagons and octagons, which may increase a contact surface area with the patient's body.

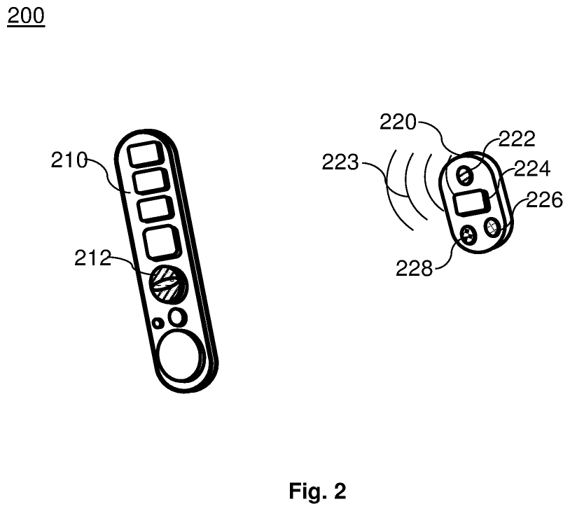

Reference is now made to FIG. 2, which is a schematic illustration of a system 200 for detecting sounds from a subject's body, according to some embodiments of the invention.

System 200 may include a device 210 for detecting sounds from the subject's body. Device 210 may be similar to device 100 described hereinabove with respect to FIGS. 1A, 1B and 1C. Device 210 may be removably attached to the subject's body to detect sounds from one or more locations within the subject's body (e.g., as described hereinabove with respect to FIGS. 1A, 1B and 1C).

System 200 may include a swallowable capsule 220. Swallowable capsule 220 may include an acoustic transducer 222. Acoustic transducer 222 may generate a sound signal 223. For example, acoustic transducer 222 may generate sound signal 223 after swallowable capsule 220 has been swallowed by the subject. In some embodiments, acoustic transducer 222 may generate sound signals of different frequencies. In some embodiments, acoustic transducer 222 may generate a series of sound signals, wherein each of the sound signals in the series may have a different frequency as compared to frequencies of other sound signals in the series.

Device 210 may detect by its acoustic sensor(s) 212 (e.g., like acoustic sensor 120 described hereinabove with respect to FIGS. 1A, 1B and 1C) sound signal 223 generated by acoustic transducer 222 of swallowable capsule 220 from within the subject's body and generate the output signal further based on the detected acoustic transducer sound. The output signal may be used for further processing, e.g., as described above with respect to FIGS. 1A, 1B and 1C. The output signal generated based on the sound signals transmitted by acoustic transducer 222 of swallowable capsule 220 from within the subject's body may, for example, provide information concerning tissues through which these sound signals have passed.

For example, acoustic transducer 222 of swallowable capsule 220 may be configured to generate a series of sound signals that may pass through the lungs of the subject. In this example, device 210 attached to the subject's body in a vicinity of the lungs may detect the sounds signals generated by acoustic transducer 222 of swallowable capsule 220 and generate respective output signal. The output signal may be analyzed to detect biomarkers indicative of, for example, obstructions that may be indicative of, for example, tumor, polypus or any other suitable condition.

In another example, acoustic transducer 222 of swallowable capsule 220 may be configured to generate a series of sound signals that may pass through the large intestine of the subject. In this example, device 210 attached to the subject's body in a vicinity of the lungs may detect the sounds signals generated by acoustic transducer 222 of swallowable capsule 220 and generate respective output signal. The output signal may be analyzed to detect biomarkers indicative of, for example, pulmonary edema, which in turn may be indicative of, for example, heart failure.

The output signals may be analyzed to, for example, determine changes within the output signals along different locations within digestion system of the subject. The analysis may, for example, include comparison of the output signals to references datasets. The reference datasets may, for example, include normal and/or abnormal sets of data values. The analysis may, for example, include utilization of artificial intelligence methods.

In some embodiments, swallowable capsule 220 may include a controller 224 configured to control acoustic transducer 222.

In some embodiments, swallowable capsule 220 may include a capsule acoustic sensor 226 configured to detect sounds from within the subject's body (e.g., sounds generated by digestive system) and generate the capsule output signal. In some embodiments, swallowable capsule 220 may include a transmitter 228 to transmit the capsule output signal. Device 210 may detect the capsule output signal by its communication unit 214 (e.g., like communication unit 136 described hereinabove with respect to FIGS. 1A, 1B and 1C). Processor 218 of device 210 (e.g., like processor 130 described hereinabove with respect to FIGS. 1A, 1B and 1C) may treat the capsule output signal similarly to the output signal(s) being generated by its acoustic sensor(s) 212 (e.g., as described hereinabove with respect to FIGS. 1A, 1B and 1C).

In some embodiments, controller 224 may control acoustic transducer 222 to continuously transmit sound signals. In some embodiments, controller 224 may control transducer 222 to transmit sound signals at specified time intervals. The specified time intervals may be, for example, predefined or dynamically updated.

In some embodiments, controller 224 may control acoustic transducer 222 to transmit sound signals when swallowable capsule reaches a target organ. The time of arrival to the target organ may be, for example, predefined based on typical digestion of the subject. In another example, controller 224 may control acoustic transducer 222 to transmit a specified signal indicating that the swallowable capsule 220 has reached the target organ.

In some embodiments, controller 224 may control acoustic transducer 222 may transmit a sound signal, acoustic sensor 226 of swallowable capsule 220 may receive reflected sound signal, and controller 224 may determine the location of swallowable capsule 220 within the digestion system of the subject based on at least one of the transmitted or reflected sound signal.

In some embodiments, controller 224 may control acoustic transducer 222 may transmit a sound signal indicating that swallowable capsule is about to leave the digestion system of the subject. In some embodiments, one or more devices 210 placed externally along gastrointestinal tract may monitor the real-time position of swallowable capsule 220 in the gastrointestinal tract.

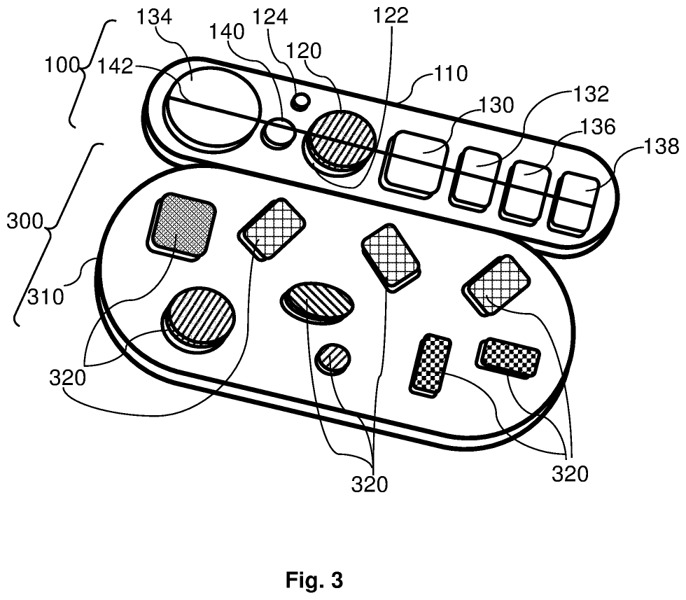

Reference is now made to FIG. 3, which is a schematic illustration of a device 100 for detecting sounds from a subject's body and an array 300 of acoustic sensors 320 connectable to device 100, according to some embodiments of the invention.

Array 300 may include a support 310 and multiple acoustic sensors 320 connected to support 310. Array 300 may include multiple acoustic waveguides (not shown), each for one of multiple acoustic sensors 320 (e.g., as described above with respect to FIGS. 1A, 1B, 1C and 1D). Support 310 may be, for example, similar to support 110 (e.g., described above with respect to FIGS. 1A, 1B, 1C and 1D). In some embodiments, support 310 of array 320 may be configured to be connected to support 110 of device 100. In some embodiments, support 110 of device 100 may be configured to be connected to support 310 of array 300. Acoustic sensors 320 of array 300 may be configured to be connected to electronic components of device 100 using a wired and/or wireless connection.

In some embodiments, acoustic sensors 320 may detect sounds of the same frequency range (e.g., the same wide frequency range or the same narrow frequency range). In some embodiments, some of acoustic sensors 320 may detect sounds of a different frequency range as compared to other acoustic sensors of acoustic sensors 320. For example, the frequency range of each of acoustic sensors 320 may be selected based on a specific organ or a subgroup of organs of the subject's body to be sensed with the respective acoustic sensor. For example, a first acoustic sensor may be capable of detecting sounds from a subject's heart and operate in a first frequency range of 20-200 Hz, a second acoustic sensor may be capable of detecting sounds from subject's lungs and operate in a second frequency range of 25-1500 Hz. In some embodiments, the wavelength ranges of acoustic sensors 320 may partly overlap with each other. In some embodiments, acoustic sensors 320 may be configured to detect sounds arriving from the same direction from within the subject's body. In some embodiments, some of acoustic sensors 320 may be configured to detect sounds arriving from a different direction from within the subject's body as compared to other acoustic sensors of acoustic sensors 320. In some embodiments, some of acoustic sensors 320 may have different shape as compared to other acoustic sensors of acoustic sensors 320. In some embodiments, some of acoustic sensors 320 may be of a different type as compared to other acoustic sensors of acoustic sensors 320.

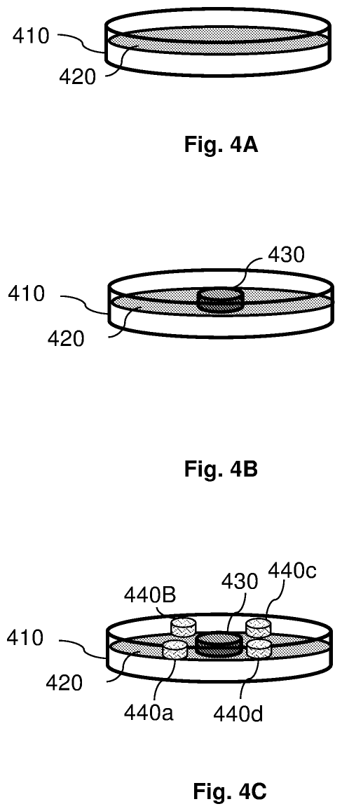

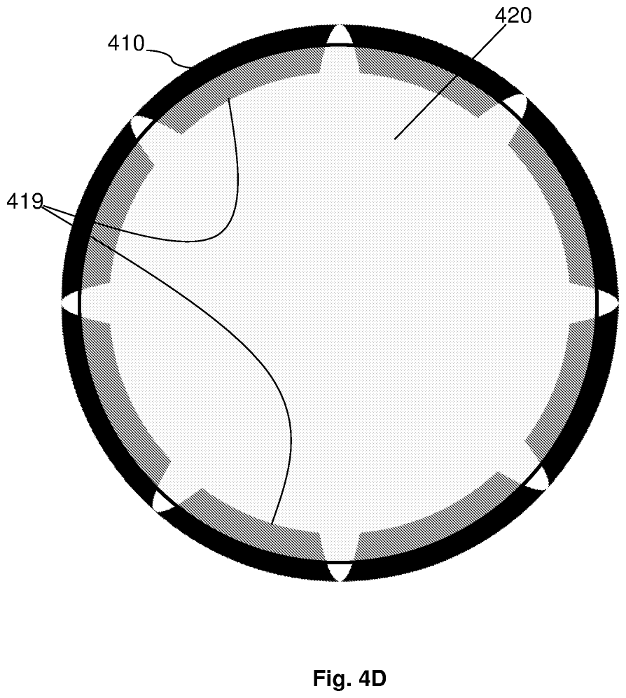

Reference is now made to FIGS. 4A-4C are schematic illustrations of a piezoelectric element 420 within a housing 410 serving as the acoustic sensor, according to some embodiments of the invention.

Reference is also made to FIG. 4D, which shows a non-limiting example of the piezoelectric plate 420 whose corners rest on supporting sections 419 of housing 410, according to some embodiments of the invention. FIG. 4D shows a schematic top view.

According to some embodiments, as shown in FIG. 4A, a piezoelectric element may be in the form of a piezoelectric plate 420 and held within housing 410 (waveguide not shown here). The piezoelectric plate 420 may be circular in shape. The piezoelectric plate may be annular. According to some embodiments the piezoelectric element is only supported in sections and not along the entirety of its perimeter. For example, FIG. 4D shows a non-limiting example of the piezoelectric plate 420 whose corners rest on supporting sections 419 of housing 410.

According to some embodiments, the piezoelectric element may be at a tension that optimizes a sensitivity of the piezoelectric element.

According to some embodiments, as shown in FIG. 4B, acoustic sensor may include a microphone 430 which can be implemented as a hydrophone. In some embodiments, acoustic sensor 120 may include a piezoelectric element 420 as explained above. For example, the piezoelectric element may include a piezoelectric film or crystal such as polyvinylidene fluoride (PVDF). In some embodiments, acoustic sensor may include a supportive case (e.g., a housing). In some embodiments, acoustic sensor may be provided without a supportive case.

According to some embodiments, the piezoelectric element may be included inside the supportive case or housing of the acoustic sensor. The housing may be configured hold or otherwise support the piezoelectric element, for example to hold the piezoelectric element at a predefined tension. The housing may be configured to comprise an internal cavity, which may allow the piezoelectric element to be displaced (e.g., vibrate) within the cavity.

According to some embodiments, as shown in FIG. 4A, a gel having desired acoustic properties may be used to enhance the acoustic coupling of acoustic sensor and acoustic waveguide to the subject's body. For example, the gel may have an acoustic impedance similar to human tissue. The gel may displace air between the subject's body and the acoustic sensor and acoustic waveguide, thereby creating a vacuum effect to improve signal acquisition. According to some embodiments, a plurality of gel pads 440a-440d may be included in the housing 410 which couples a piezoelectric element 420 and possibly microphone 430 to the housing 410.

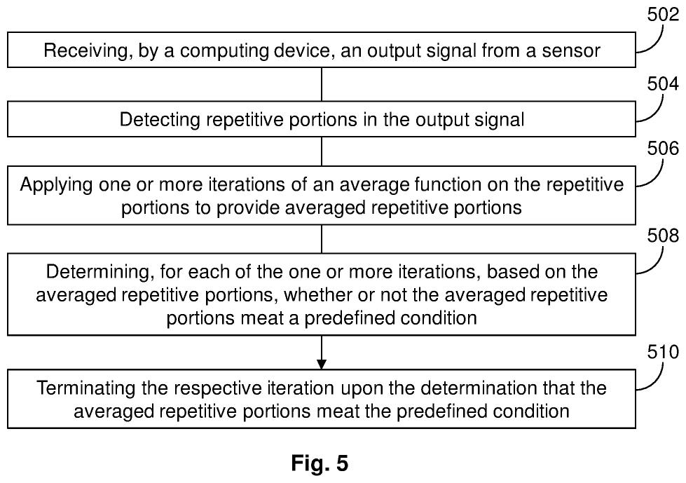

Reference is now made to FIG. 5, which is a flowchart of a method of averaging a signal, according to some embodiments of the invention.

Operations described with respect to FIG. 5 may be performed by processor 130 of device 100 described hereinabove, computing device 1100 described hereinbelow and/or by any other suitable computing device and/or a combination of devices.

The method may include receiving 502, by a computing device, an output signal being generated by a sensor. For example, the sensor may be an acoustic sensor of a device for recording and detecting sounds from a subject's body, such as device 100 described hereinabove. The output signal may be indicative of sounds being detected by the acoustic sensor from within the subject's body. The output signal may be periodic or quasiperiodic. The output signal may be non-periodic. The output signal may include periodic (e.g., repetitive) portions and non-periodic (e.g., no repetitive) portions. For example, if the output signal is indicative of sounds detected from the subject's heart and more generally from the subject's cardiovascular system, the output signal may include repetitive portions indicative of sounds generated by heart beats (valves' sounds S1 S2) or by heart valve disease of the subject's and non-repetitive portions generated due to, for example, irregular/chaotic heart beats or artery/aortic stenosis. In another example, if the output signal is indicative of sounds detected from the subject's lungs, the output signal may include repetitive portions indicative of sounds generated by breathing of the subject's and non-repetitive portions generated due to, for example, pulmonary edema, hypotension or hypertension. In another example, if the output signal is indicative of sounds detected from the fetus developing in the subject's uterus, the output signal may include repetitive portions indicative of sounds generated by, for example, fetal heart and non-repetitive portions indicative of sounds generated by, for example, fetal motions or uterine contractions. The computing device may be, for example, a processor of device 100 or any external computing device. For example, the communication unit of device 100 may transmit the output signal or its derivatives to the computing device.

The method may include detecting 504 repetitive portions in the output signal.

The method may include applying 506 one or more iterations of an average function on the repetitive portions to provide averaged repetitive portions.

The method may include determining 508, for each of the one or more iterations, based on the averaged repetitive portions, whether or not the averaged repetitive portions meet a predefined condition.

The method may include terminating 510 the respective interaction upon the determination that the averaged repetitive portions meet the predefined condition.

Some embodiments may include determining, for each of the one or more iterations, a signal to noise ratio (SNR) in the averaged repetitive portions of the output signal and terminating the respective iteration if the SNR has reached a specified SNR value.

Some embodiments may include determining, for each of the one or more iterations, a number of the repetitive portions or the averaged repetitive portions in the output signal and terminating the respective iteration if the number of the repetitive portions or the averaged repetitive portions has reached a specified number of repetitive portions. The specified number of repetitive portions may be, for example, preset or may be determined based on an average number of repetitive portions in the output signal over a specified time interval (e.g., over an hour).

Some embodiments may include determining, for each of the one or more iterations, a cross-correlation value between the averaged repetitive portions and a reference signal and terminating the respective iteration if the cross-correlation value has reached a specified cross-correlation value. The specified cross-correlation value may be, for example, preset or may be determined based on an average cross-correlation value in the output signal over a specified time interval (e.g., over an hour).

In some embodiments, each of the averaged repetitive portions includes a first section having data values that are above a preset value and a second section having data values that are below the present value. For example, if the output signal is indicative of sounds generated by the subject's heart, the first section of the averaged repetitive portion may include data values relating to a systole portion of the heartbeat cycle and the second section of the averaged repetitive portion may include data values relating to a diastole portion of the heartbeat cycle. Some embodiments may include determining, for each of the one or more iterations, a SNR value in the second sections of the averaged repetitive portions and terminating the respective iteration if the SNR value in the second sections of the averaged repetitive portions has reached a specified SNR value. The specified SNR value may be, for example, preset or may be determined based on an average SNR value in the output signal over a specified time interval (e.g., over an hour).

Some embodiments may include applying the average function on a specified number of the repetitive portions of the output signal. The specified number of the repetitive portions may be, for example, preset or determined based on a preset SNR value.

The method disclosed herein with respect to FIG. 5 may, for example, provide adaptive averaging of the signal and/or enhance the SNR of the signal. The method may limit the number of averaging iterations to a minimum required to enhance the SNR of the signal. Limiting the number of averaging iterations to a minimum may, for example, save power consumption of the device performing the method (e.g., device 100 described hereinabove). Limiting the number of averaging iterations to a minimum may, for example, make sure that important data in the signal is not being smoothed by over averaging.

Some embodiments may include determining, based on the averaged repetitive portions, one or more subsets of data values indicative of one or more sound patterns being detected from within the subject's body. For example, normal sound pattern(s) and/or abnormal/pathological sound patterns may be determined. Some embodiments may include generating a notification (e.g., visual, sound and/or mechanical notification) indicative of the one or more sound patterns detected from within the subject's body. Some embodiments may include transmitting the notification to a remote computing device and/or an alarming (e.g., remote alarming) device.

Various embodiments may include analyzing the averaged repetitive portions and/or the one or more sound patterns to determine one or more biomarkers indicative of a health/physical/fitness-related/wellness-related condition of the subject. For example, normal biomarker(s) and/or abnormal/pathological biomarker(s) may be determined. For example, the biomarker(s) in the output signal(s) may be analyzed using one or more pre-trained artificial intelligence (AI) models and/or pre-trained machine learning models. Some embodiments may include generating a notification (e.g., visual, sound and/or mechanical notification) indicative of the one or more biomarkers indicative of the health/physical/fitness-related/wellness-related condition of the subject. Some embodiments may include transmitting the notification to a remote computing device and/or an alarming (e.g., remote alarming) device.

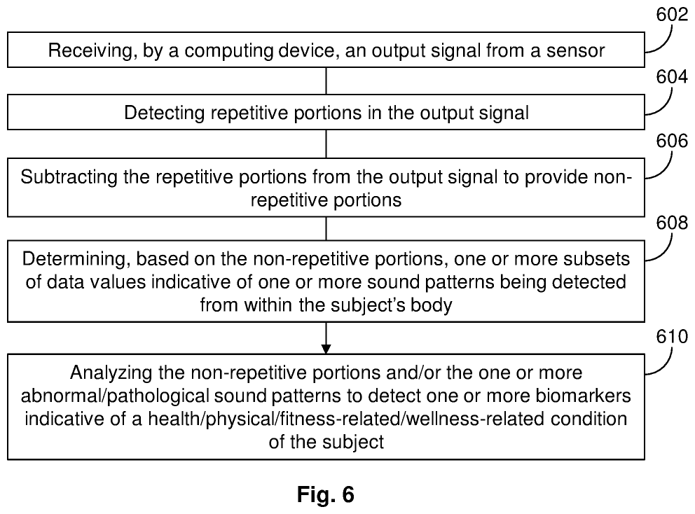

Reference is now made to FIG. 6, which is a flowchart of a method of analyzing a signal, according to some embodiments of the invention.

Operations described with respect to FIG. 6 may be performed by processor 130 of device 100 described hereinabove, computing device 1100 described hereinbelow and/or by any other suitable computing device and/or a combination of devices.

The method may include receiving 602, by a computing device, an output signal being generated by a sensor. For example, the sensor may be an acoustic sensor of a device for recording and detecting sounds from a subject's body, such as device 100 described hereinabove. The output signal may be indicative of sounds being detected by the acoustic sensor from within the subject's body. The output signal may be periodic or quasiperiodic. The output signal may be non-periodic. The output signal may include periodic (e.g., repetitive) portions and non-periodic (e.g., no repetitive) portions (e.g., as described above with respect to FIG. 5). The computing device may be, for example, a processor of device 100 or any external computing device such as computing device 1100 described hereinbelow. For example, the communication unit of device 100 may transmit the output signal or its derivatives to the computing device.

The method may include detecting 604 repetitive portions in the output signal.

The method may include subtracting 606 the repetitive portions from the output signal to provide non-repetitive portions.

Some embodiments may include determining 608, based on the non-repetitive portions, one or more subsets of data values indicative of one or more sound patterns being detected from within the subject's body. For example, normal sound patterns and/or abnormal/pathological sound patterns may be determined. Some embodiments may include generating a notification (e.g., visual, sound and/or mechanical notification) indicative of the one or more sound patterns detected from within the subject's body. Some embodiments may include transmitting the notification to a remote computing device and/or an alarming (e.g., remote alarming) device.

Various embodiments may include analyzing 610 the non-repetitive portions and/or the one or more abnormal/pathological sound patterns to determine one or more biomarkers indicative of a health/physical/fitness-related/wellness-related condition of the subject. For example, normal biomarkers and/or abnormal/pathological biomarkers may be determined. Some embodiments may include generating a notification (e.g., visual, sound and/or mechanical notification) indicative of the one or more biomarkers indicative of the health/physical/fitness-related/wellness-related condition of the subject. Some embodiments may include transmitting the notification to a remote computing device and/or an alarming (e.g., remote alarming) device.

Some embodiments may include applying one or more iterations of an average function on the non-repetitive portions to provide averaged non-repetitive portions (e.g., as described above with respect to FIG. 6). Some embodiments may include determining, for each of the one or more iterations, based on the averaged non-repetitive portions, whether or not the averaged non-repetitive portions meet a predefined condition (e.g., as described above with respect to FIG. 6). Some embodiments may include terminating the respective interaction upon the determination that the averaged non-repetitive portions meet the predefined condition (e.g., as described above with respect to FIG. 6).

Some embodiments may include determining, based on the averaged non-repetitive portions, the one or more subsets of data values indicative of one or more sound patterns being detected from within the subject's body.

Various embodiments may include analyzing the averaged non-repetitive portions and/or the one or more abnormal/pathological sound patterns to detect the one or more biomarkers indicative of a health/physical/fitness-related/wellness-related condition of the subject.

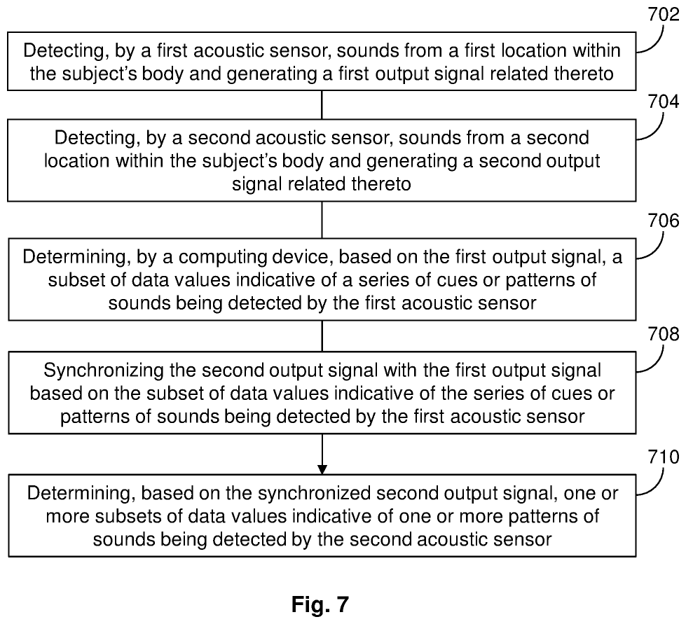

Reference is now made to FIG. 7, which is a flowchart of a method of detecting and analyzing sounds from within two or more locations within a subject's body, according to some embodiments of the invention.

Operations described with respect to FIG. 7 may be performed by processor 130 of device 100 described hereinabove, computing device 1100 described hereinbelow and/or by any other suitable computing device and/or a combination of devices.

The method may include detecting 702, by a first acoustic sensor, sounds from a first location within the subject's body and generating a first output signal related thereto. The method may include detecting 704, by a second acoustic sensor, sounds from a second location within the subject's body and generating a second output signal related thereto. In some embodiments, one or more devices for recording and detecting sounds from a subject's body, such as device 100 described hereinabove, may be used to detect sounds from within the first location and the second location within the subject's body. For example, a first device (e.g., like device 100) having the first acoustic sensor and a second device (e.g., like device 100) having the second acoustic sensor may be placed (e.g., as described hereinabove) in a vicinity of the first location and the second location to detect sounds. In another example, a single device (e.g., like device 100) having the first acoustic sensor and the second acoustic sensor may be used. The first output signal and the second output signal may be periodic or quasiperiodic.

Some embodiments may include detecting the sounds from within the first location and the second location within the subject's body over a specified period of time. The specified period of time may range within, for example, few seconds to few months, few hours to weeks, few hours to days or any other suitable range. The sounds may be, for example, continuously detected during the specified period of time. In another example, the sounds may be detected in two or more time-separated sessions during the specified period of time.

One advantage of using device(s) 100 for recording and detecting sounds is that device(s) 100 (i) having small dimensions and weight, (ii) requiring small contact pressure (or requires no contact pressure at all) to efficiently guide the sounds detected from within the subject's body to the acoustic sensor, and (iii) being attached to the subject's body by simple means (e.g., using sticky flat flexible support, for example as described hereinabove. Accordingly, device(s) 100 may remain attached to the subject's body for long periods of times (e.g., days, weeks, months, etc.) without causing (or substantially without causing) inconvenience to the subject. This in contrast to typical commercial electronic stethoscope devices that are larger and heavier than device(s) 100 and thus cannot practically remain attached the subject's body for long periods of times of few days, weeks, months, but rather may be used for spot checks only.

The method may include determining 706, by a computing device, based on the first output signal, a subset of data values indicative of a series of cues or patterns of sounds being detected by the first acoustic sensor. The computing device may be, for example, a processor of device(s) 100 (e.g., as described hereinabove) or any other suitable computing device external to device(s) 100 (e.g., such as computing device 1100 described hereinbelow). The method may include synchronizing 708 the second output signal with the first output signal based on the subset of data values indicative of the series of sound cues or sounds patterns being detected from the first location within the subject's body. For example, the first output signal may be periodic or quasiperiodic and having detectable periodicity, e.g., due to sufficiently high SNR value of the first output signal. In this example, the second output signal may be periodic or quasiperiodic and having undetectable periodicity, e.g., due to insufficient SNR value of the second output signal. Synchronization of the second output signal based on the sound cues or sounds patterns being detected from the first location within the subject's body may enable detecting the periodicity or quasi-periodicity of the second output signal. Detection of the periodicity or quasi-periodicity of the second output signal may enable processing (e.g., averaging as described above with respect to FIG. 6) and/or analysis of the second output signal. The method may include determining 710, based on the synchronized second output signal, one or more subsets of data values indicative of one or more patterns of sound being detected by the second acoustic sensor. For example, normal sound patterns and/or abnormal/pathological sounds patterns may be determined.

Some embodiments may include determining, based on the first output signal, one or more subsets of data values indicative of one or more patterns of sounds being detected by the first acoustic sensor. For example, normal sound patterns and/or abnormal/pathological sounds patterns may be determined. Some embodiments may include generating a notification (e.g., visual, sound and/or mechanical notification) indicative of the one or more sound patterns detected from within the subject's body. Some embodiments may include transmitting the notification to a remote computing device and/or an alarming (e.g., remote alarming) device.

Various embodiments may include analyzing the first output signal and/or the one or more patterns of sounds being detected by the first acoustic sensor to determine one or more biomarkers indicative of a health/physical/fitness-related/wellness-related condition of the subject. For example, normal biomarkers and/or abnormal/pathological biomarkers may be determined. Some embodiments may include generating a notification (e.g., visual, sound and/or mechanical notification) indicative of the one or more biomarkers indicative of the health/physical/fitness-related/wellness-related condition of the subject. Some embodiments may include transmitting the notification to a remote computing device and/or an alarming (e.g., remote alarming) device.

Various embodiments may include analyzing the synchronized second output signal and/or the one or more patterns of sound being detected by the second acoustic sensor to detect one or more biomarkers indicative of a health/physical/fitness-related/wellness-related condition of the subject. For example, normal biomarkers and/or abnormal/pathological biomarkers may be determined.

Some embodiments may include determining a correlation between (i) the one or more patterns of sounds being detected by the first acoustic sensor and (ii) the one or more patterns of sound being detected by the second acoustic sensor. Some embodiments may include determining, based on the correlation, one or more biomarkers indicative of a health/physical/fitness-related/wellness-related condition of the subject. For example, normal biomarkers and/or abnormal/pathological biomarkers may be determined.

The first acoustic sensor and the second acoustic sensor may be placed on or in a vicinity to the subject's body to detect sounds, for example, from the cardiovascular system of the subject.

In one example, the first acoustic sensor may detect sounds from at least a portion of the subject's heart (e.g., ventricles, valves or any other suitable portion) and the sound cues/patterns being detected may, for example, include sound cues/patterns being generated by mechanical activity of at least a portion of the subject's heart including, e.g., contraction, motion of valves, blood flow or any other suitable mechanical activity. In this example, the second acoustic sensor may detect sounds from one or more arteries or veins of the subject. In this example, the first output signal and the second output signal may be synchronized and analyzed to determine sound patterns and/or biomarkers indicative of, for example, blood flow related conditions/disorders of the subject.

The first acoustic sensor and the second acoustic sensor may be placed on or in a vicinity to the subject's body to detect sounds, for example, from the digestive system of the subject. For example, the first acoustic sensor and the second acoustic sensors may detect sounds from one of the subject's stomach, large intestine, small intestine, esophagus or any other suitable location within the subject's digestive system.

In one example, the first acoustic sensor may detect sounds from the first location along the large intestine and the second acoustic sensor may detect sounds from the second location along the large intestine of the subject. In this example, the first output signal and the second output signal may be synchronized and analyzed to determine sound patterns and/or biomarkers indicative of, for example, obstructions that may be indicative of, for example, tumor, polypus or any other suitable condition.

The first acoustic sensor and the second acoustic sensor may be placed on or in a vicinity to the subject's body to detect sounds, for example, from the subject's heart, arteries, veins, lungs, trachea, larynx, pharynx, diaphragm, bronchus, bronchiole, nose, apnea, liver, kidney, pancreas, uterus, vagina, fallopian tubes, fetus, fetus, fetal heart, joints or any other suitable organ or combination of organs generating sounds due to mechanical, metabolic or any other suitable activity. For example, the first acoustic sensor and the second acoustic sensor may detect sounds from the same organ within the subject's body. In another example, the first acoustic sensor may detect sounds from the first organ and the second acoustic sensor may detect sounds from the second organ within the subject's body.

In one example, the first acoustic sensor may detect sounds from at least a portion of the subject's heart and the second acoustic sensor may detect sounds from at least a portion of the lungs of the subject. In this example, the first output signal and the second output signal may be synchronized and analyzed to determine sound patterns and/or biomarkers indicative of, for example, congestive heart failure (CHF), pulmonary edema or any other suitable condition.

Some embodiments may include measuring, by a third sensor, a parameter of the subject's body and generating a third output signal related thereto. The third sensor may be non-acoustic sensor. The third sensor may be, for example, optical, electrical, chemical or any other suitable sensor. Some embodiments may include determining, based on the third output signal, one or more subsets of data values indicative of one or more parameter patterns being measured by the third sensor. Some embodiments may include determining a correlation between at least one of (i) the one or more patterns of sounds being detected by the first acoustic sensor and (ii) the one or more patterns of sound being detected by the second acoustic sensor, (iii) the one or more parameter patterns being measured by the third sensor, (iv) or any combination thereof. Some embodiments may include determining, based on the correlation, one or more biomarkers indicative of a health/physical/fitness-related/wellness-related condition of the subject. For example, normal biomarkers and/or abnormal/pathological biomarkers may be determined.

In one example, the first acoustic sensor may detect sounds from at least a portion of the subject's heart and the second acoustic sensor may detect sounds from at least a portion of the lungs, and the third sensor may be an oxygen saturation sensor that may measure blood oxygen saturation of the subject. In this example, the first output signal, the second output signal and the third output signal may be synchronized and analyzed to determine sound patterns and/or biomarkers indicative of, for example, infection in cardiovascular and/or pulmonary systems of the subjects (e.g., such as COVID-19 or any other suitable disease).

In some embodiments, the first output signal, the second output signal and, optionally, the third output signal may be synchronized and analyzed to determine new sound patterns and/or new biomarkers indicative of a health/physical/fitness-related/wellness-related condition of the subject.

In some embodiments, the first acoustic sensor and the second acoustic sensor may detect sounds from symmetric organs within the subject's body. For example, the first acoustic sensor may detect sounds from the first lung of the subject and the second acoustic sensor may detect sounds from the second lung of the subject. The first output signal and the second output may be synchronized and analyzed to determine sound patterns and/or biomarkers indicative of a health/physical/fitness-related/wellness-related condition of the subject relating to, for example, the symmetric organs.

Some embodiments may include instructing the subject to change the location of the first acoustic sensor and/or of the second acoustic sensor and/or to add one or more additional acoustic sensors as part of a predefined protocol and/or based on the determined sound patterns and/or determined biomarkers.

Some embodiments may include detecting ambient sounds. For example, the ambient sounds may be detected by a microphone or the first acoustic sensor and/or the second acoustic sensor prior to placing the sensors thereof on or in a vicinity to the subject's body. The microphone may be a microphone of the subject's smartphone, voice assistant device, wearable microphone or any other suitable microphone device. Some embodiments may include filtering the ambient sounds from the first output signal and/or the second output signal. Filtering of the ambient sounds from the first output signal and/or the second output signal may, for example, improve the SNR of the respective signal.

While two acoustic sensors being described, three or more sensors may be used to detect and analyze sounds from three or more different locations and/or directions within the subject's body.

The disclosed method may, for example, enable determining sound patterns and/or biomarkers indicative of a health/physical/fitness-related/wellness-related condition of the subject and/or determining new sound patterns and/or new biomarkers related thereto. The disclosed method may, for example, overcome the disadvantages of low SNR of sound related signals being detected from within the subject's body due to, for example: (i) using multiple acoustic sensors, and (ii) analyzing sound related signals being detected and recorded for long periods of time (e.g., hours, days, weeks, months, etc.) by enabling ignoring/filtering out momentary events from the signals.

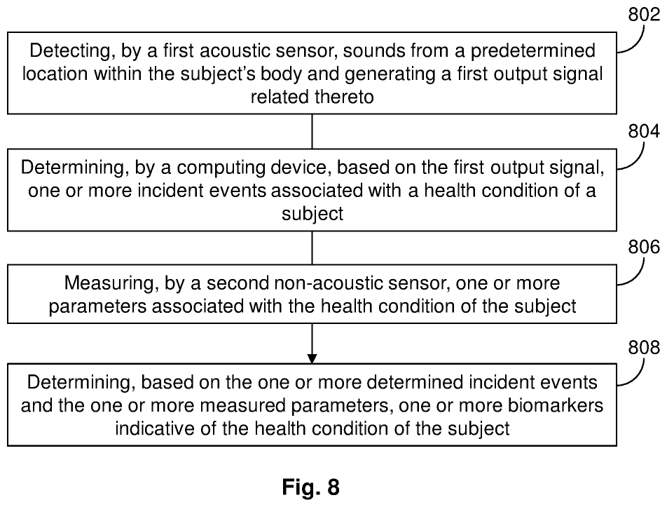

Reference is now made to FIG. 8, which is a flowchart of a method of determining one or more biomarkers indicative of a health condition of a subject based on an acoustic sensor and a non-acoustic sensor, according to some embodiments of the invention.

Operations described with respect to FIG. 8 may be performed by processor 130 of device 100 described hereinabove, computing device 1100 described hereinbelow and/or by any other suitable computing device and/or a combination of devices.

The method may include detecting 802, by a first acoustic sensor, sounds from a predetermined location within the subject's body and generating a first output signal related thereto. For example, the first acoustic sensor may be an acoustic sensor of a device for recording and detecting sounds from a subject's body, such as device 100 described hereinabove.

The method may include determining 804, by a computing device, based on the first output signal, one or more incident events associated with a health condition of a subject. The computing device may be, for example, a processor of device 100 (e.g., as described hereinabove) or any other suitable computing device external to device 100 (e.g., such as computing device 1100 described hereinbelow).

The method may include measuring 806, by a second non-acoustic sensor, one or more parameters associated with the health condition of the subject.

The method may include determining 808, based on the one or more determined incident events and the one or more measured parameters, one or more biomarkers indicative of the health condition of the subject. Some embodiments may include generating a notification (e.g., visual, sound and/or mechanical notification) indicative of the one or more biomarkers indicative of the health condition of the subject. Some embodiments may include transmitting the notification to a remote computing device and/or an alarming (e.g., remote alarming) device.

Some embodiments may include determining, based on the one or more incident events and the one or more measured parameters, a cumulative load of the incident events. Some embodiments may include generating a notification (e.g., visual, sound and/or mechanical notification) indicative of the cumulative load of the incident events. Some embodiments may include transmitting the notification to a remote computing device and/or an alarming (e.g., remote alarming) device.

For example, the first acoustic sensor may detect sounds from at least a portion of the subject's heart. The incident event may, for example, include atrial fibrillation (AF). The second sensor may, for example, measure a concentration of plasma lactate of the subject. The health condition of the subject may, for example, include a cumulative load of AF of the subject.

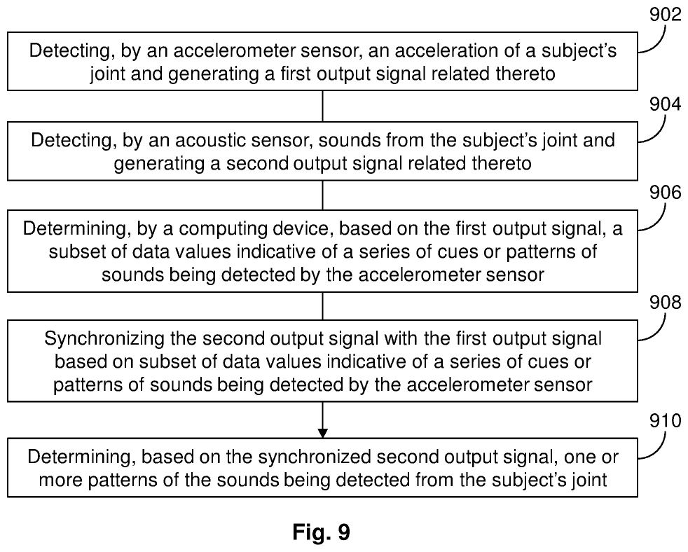

Reference is now made to FIG. 9, which is a flowchart of a method of detecting and analyzing sounds from a subject's joint, according to some embodiments of the invention.

Operations described with respect to FIG. 9 may be performed by processor 130 of device 100 described hereinabove, computing device 1100 described hereinbelow and/or by any other suitable computing device and/or a combination of devices.

The method may include detecting 902, by an accelerometer sensor, an acceleration of a subject's joint and generating a first output signal related thereto. The method may include detecting 804, by an acoustic sensor, sounds from the subject's joint and generating a second output signal related thereto. For example, the accelerometer sensor and the acoustic sensors may be sensors of a device for recording and detecting sounds from a subject's body, such as device 100 described hereinabove.

The method may include determining 906, by a computing device, based on the first output signal, a subset of data values indicative of a series of cues or patterns of sounds being detected by the accelerometer sensor.

The method may include synchronizing 908 the second output signal with the first output signal based on subset of data values indicative of a series of cues or patterns of sounds being detected by the accelerometer sensor.

The method may include determining 910, based on the synchronized second output signal, one or more patterns of the sounds being detected from the subject's joint.

Some embodiments may include determining, based on at least one of the first output signal, the synchronized second output signal, one or more determined patterns of the sounds or any combination thereof, one or more biomarkers indicative of the health condition of the subject. Some embodiments may include generating a notification (e.g., visual, sound and/or mechanical notification) indicative of the one or more biomarkers indicative of the health condition of the subject. Some embodiments may include transmitting the notification to a remote computing device and/or an alarming (e.g., remote alarming) device.

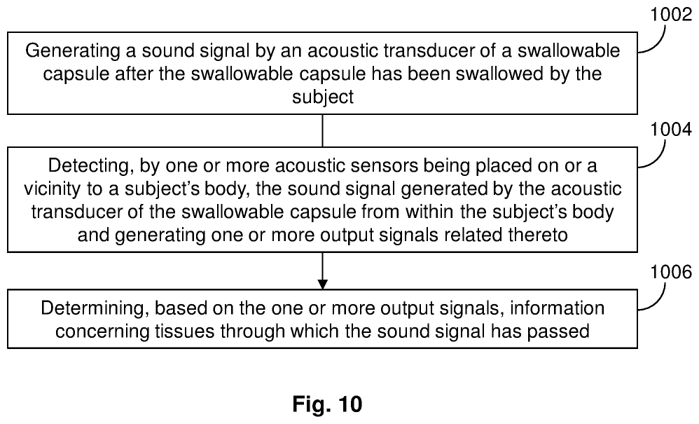

Reference is now made to FIG. 10, which is a flowchart of a method of detecting sounds from a subject's body based on an external acoustic sensor and a swallowable capsule, according to some embodiments of the present invention.

Operations described with respect to FIG. 10 may be performed by processor 130 of device 100 described hereinabove, computing device 1100 described hereinbelow and/or by any other suitable computing device and/or a combination of devices and/or a combination of devices.

The method may be performed by, for example, system 200 described hereinabove.

The method may include generating 1002 a sound signal by an acoustic transducer of a swallowable capsule after the swallowable capsule has been swallowed by the subject. For example, the acoustic transducer and the swallowable capsule may be acoustic transducer 222 and swallowable capsule 220 of system 200 described hereinabove.

The method may include detecting 1004, by one or more acoustic sensors being placed on or a vicinity to a subject's body, the sound signal generated by the acoustic transducer of the swallowable capsule from within the subject's body and generating one or more output signals related thereto.

The method may include determining 1006, based on the one or more output signals, information concerning tissues through which the sound signal has passed (e.g., as described above with respect to FIG. 2). Some embodiments may include generating a notification (e.g., visual, sound and/or mechanical notification) indicative of the, information concerning tissues through which the sound signal has passed. Some embodiments may include transmitting the notification to a remote computing device and/or an alarming (e.g., remote alarming) device.

Reference is now made to FIG. 11, which is a block diagram of an exemplary computing device 1100 which may be used with embodiments of the present invention.

Computing device 1100 may include a controller or processor 1105 that may be, for example, a central processing unit processor (CPU), a chip or any suitable computing or computational device, an operating system 1115, a memory 1120, a storage 1130, input devices 1135 and output devices 1140.

Operating system 1115 may be or may include any code segment designed and/or configured to perform tasks involving coordination, scheduling, arbitration, supervising, controlling or otherwise managing operation of computing device 1100, for example, scheduling execution of programs. Memory 1120 may be or may include, for example, a Random Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 1120 may be or may include a plurality of, possibly different, memory units. Memory 1120 may store for example, instructions to carry out a method (e.g., code 1125), and/or data such as user responses, interruptions, etc.

Executable code 1125 may be any executable code, e.g., an application, a program, a process, task or script. Executable code 1125 may be executed by controller 1105 possibly under control of operating system 1115. In some embodiments, more than one computing device 1100 or components of device 1100 may be used for multiple functions described herein. For the various modules and functions described herein, one or more computing devices 1100 or components of computing device 1100 may be used. Devices that include components similar or different to those included in computing device 1100 may be used, and may be connected to a network and used as a system. One or more processor(s) 1105 may be configured to carry out embodiments of the present invention by for example executing software or code. Storage 1130 may be or may include, for example, a hard disk drive, a floppy disk drive, a Compact Disk (CD) drive, a CD-Recordable (CD-R) drive, a universal serial bus (USB) device or other suitable removable and/or fixed storage unit. In some embodiments, some of the components shown in FIG. 9 may be omitted.

Input devices 1135 may be or may include a mouse, a keyboard, a touch screen or pad or any suitable input device. It will be recognized that any suitable number of input devices may be operatively connected to computing device 1100 as shown by block 1135. Output devices 1140 may include one or more displays, speakers and/or any other suitable output devices. It will be recognized that any suitable number of output devices may be operatively connected to computing device 1100 as shown by block 1140. Any applicable input/output (I/O) devices may be connected to computing device 1100, for example, a wired or wireless network interface card (NIC), a modem, printer or facsimile machine, a universal serial bus (USB) device or external hard drive may be included in input devices 1135 and/or output devices 1140.

Embodiments of the invention may include one or more article(s) (e.g., memory 1120 or storage 1130) such as a computer or processor non-transitory readable medium, or a computer or processor non-transitory storage medium, such as for example a memory, a disk drive, or a USB flash memory, encoding, including or storing instructions, e.g., computer-executable instructions, which, when executed by a processor or controller, carry out methods disclosed herein.

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the foregoing detailed description, numerous specific details are set forth in order to provide an understanding of the invention. However, it will be understood by those skilled in the art that the invention can be practiced without these specific details. In other instances, well-known methods, procedures, and components, modules, units and/or circuits have not been described in detail so as not to obscure the invention. Some features or elements described with respect to one embodiment can be combined with features or elements described with respect to other embodiments.

Although embodiments of the invention are not limited in this regard, discussions utilizing terms such as, for example, “processing,” “computing,” “calculating,” “determining,” “establishing”, “analyzing”, “checking”, or the like, can refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that can store instructions to perform operations and/or processes.

Although embodiments of the invention are not limited in this regard, the terms “plurality” and “a plurality” as used herein can include, for example, “multiple” or “two or more”. The terms “plurality” or “a plurality” can be used throughout the specification to describe two or more components, devices, elements, units, parameters, or the like. The term set when used herein can include one or more items. Unless explicitly stated, the method embodiments described herein are not constrained to a particular order or sequence. Additionally, some of the described method embodiments or elements thereof can occur or be performed simultaneously, at the same point in time, or concurrently.

In the above description, an embodiment is an example or implementation of the invention. The various appearances of “one embodiment”, “an embodiment”, “certain embodiments” or “some embodiments” do not necessarily all refer to the same embodiments. Although various features of the invention can be described in the context of a single embodiment, the features can also be provided separately or in any suitable combination. Conversely, although the invention can be described herein in the context of separate embodiments for clarity, the invention can also be implemented in a single embodiment. Certain embodiments of the invention can include features from different embodiments disclosed above, and certain embodiments can incorporate elements from other embodiments disclosed above. The disclosure of elements of the invention in the context of a specific embodiment is not to be taken as limiting their use in the specific embodiment alone. Furthermore, it is to be understood that the invention can be carried out or practiced in various ways and that the invention can be implemented in certain embodiments other than the ones outlined in the description above.

The invention is not limited to those diagrams or to the corresponding descriptions. For example, flow need not move through each illustrated box or state, or in exactly the same order as illustrated and described. Meanings of technical and scientific terms used herein are to be commonly understood as by one of ordinary skill in the art to which the invention belongs, unless otherwise defined. While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

1. A method of analyzing a signal indicative of sounds detected from within a subject's body, the method comprising, using a computing device operating a processor:
receiving an output signal generated by a sensor, the output signal being indicative of sounds detected from within a subject's body;
detecting repetitive portions in the output signal;
subtracting the repetitive portions from the output signal to provide non-repetitive portions; and
determining, based on the non-repetitive portions, one or more subsets of data values indicative of one or more abnormal/pathological sound patterns detected from within the subject's body.

2. The method of claim 1, comprising:
based on at least one of the non-repetitive portions and the one or more abnormal/pathological sound patterns, detecting one or more biomarkers indicative of a health/physical/fitness-related/wellness-related condition of the subject.

3. The method of claim 1, further comprising:
applying one or more iterations of an average function on the non-repetitive portions to provide averaged non-repetitive portions;
determining, for each of the one or more iterations, based on the averaged non-repetitive portions, whether or not the averaged non-repetitive portions meet a predefined condition; and
terminating the respective interaction upon the determination that the averaged non-repetitive portions meet the predefined condition.

4. The method of claim 3, comprising:
determining the one or more subsets of data values indicative of the one or more abnormal/pathological sound patterns based on the averaged non-repetitive portions.

5. The method of claim 1, comprising:
detecting the one or more biomarkers indicative of the health/physical/fitness-related/wellness-related condition of the subject based on at least one of the averaged non-repetitive portions and the one or more abnormal/pathological sound patterns.

6. The method of claim 1, comprising:
applying one or more iterations of an average function on the repetitive portions to provide averaged repetitive portions;
determining, for each of the one or more iterations, based on the averaged repetitive portions, whether or not the averaged repetitive portions meet a condition; and
terminating the respective interaction upon the determination that the averaged repetitive portions meet the predefined condition.

7. The method of claim 6, comprising:
determining, for each of the one or more iterations, a signal to noise ratio (SNR) value in the averaged repetitive portions of the output signal and terminating the respective iteration if the SNR value has reached a specified SNR value.

8. The method of claim 6, further comprising:
determining, for each of the one or more iterations, a number of the repetitive portions or the averaged repetitive portions in the output signal, and
terminating the respective iteration if the number of the repetitive portions or the averaged repetitive portions has reached a specified number of repetitive portions.

9. The method of claim 8, wherein the specified number of repetitive portions is preset or determined based on an average number of repetitive portions in the output signal over a specified time interval.

10. The method of claim 6, further comprising:
determining, for each of the one or more iterations, a cross-correlation value between the averaged repetitive portions and a reference signal, and
terminating the respective iteration if the cross-correlation value has reached a specified cross-correlation value.

11. The method of claim 10, wherein the specified cross-correlation value is preset or determined based on an average cross-correlation value in the output signal over a specified time interval.

12. The method of claim 6, wherein:
each of the averaged repetitive portions includes a first section having data values that are above a preset value and a second section having data values that are below the present value, and
wherein the method further comprising:
determining, for each of the one or more iterations, a SNR value of the second sections of the averaged repetitive portions, and
terminating the respective iteration if the SNR value of the second sections of the averaged repetitive portions has reached a specified SNR value.

13. The method of claim 12, wherein the specified SNR value is preset or determined based on an average SNR value in the output signal over a specified time interval.

14. The method of claim 6, further comprising applying the average function on a specified number of the repetitive portions of the output signal.

15. The method of claim 14, wherein the specified number of the repetitive portions is preset or determined based on a preset SNR value.

16. The method of claim 6, further comprising at least one of:
determining the one or more subsets of data values indicative of the one or more abnormal/pathological sound patterns based on the averaged repetitive portions.

17. The method of claim 6, further comprising detecting one or more abnormal/pathological biomarkers indicative of a health/physical/fitness-related/wellness-related condition of the subject based on at least one of the averaged repetitive portions and the one or more abnormal/pathological sound patterns.

18. The method of claim 1, comprising generating a notification indicative of the one or more sound patterns detected from within the subject's body.

19. The method of claim 1, comprising transmitting a notification indicative of the one or more sound patterns detected from within the subject's body to a remote device.

20. The method of claim 2, comprising generating a notification indicative of the one or more biomarkers indicative of the health/physical/fitness-related/wellness-related condition of the subject.

21.-39. (canceled)
